(19) United States
(12) Patent Application Publication
(10) Pub. No.: US 2025/0002366 A1
(43) Pub. Date: Jan. 2, 2025

(54) ZINC OXIDE AND ALUMINIUM OXIDE CONTAINING MATERIALS

(71) Applicant: Advance ZincTek Limited, Rocklea, Queensland (AU)

(72) Inventor: Lev Mizikovsky, Rocklea (AU)

(21) Appl. No.: 18/710,100

(22) PCT Filed: Nov. 16, 2022
(86) PCT No.: PCT/AU2022/051369

§ 371 Date: May 14, 2024

(30) Foreign Application Priority Data
Nov. 16, 2021 (AU) 2021903680

(51) Int. Cl. C01G 9/02; A61K 8/26; A61K 8/27; A61Q 17/04; C01F 7/441; C09C 1/00

(52) U.S. Cl. CPC C01G 9/02; A61K 8/26; A61K 8/27; A61Q 17/04; C01F 7/441; C09C 1/0081; C01P 2002/54; C01P 2002/85; C01P 2004/03; C01P 2004/20; C01P 2004/61; C01P 2004/62; C01P 2004/82; C01P 2006/14; C01P 2006/60

(57) ABSTRACT

Disclosed herein are zinc oxide and aluminium oxide containing materials, particularly calcinated mixtures comprising aluminium oxide and zinc oxide, and methods for making the same.

### CROSS REFERENCE

The present application claims priority to Australian provisional application number 2021903680, filed 16 Nov. 2021, the entire contents of which is incorporated herein by reference in its entirety.

### FIELD OF THE INVENTION

The invention relates to zinc oxide and aluminium oxide containing materials and methods for making the same. In particular, the invention relates to zinc oxide and aluminium oxide containing materials that may have improved UV-absorbing, UV-visible light scattering, and/or UV-visible light reflecting properties. However, it will be appreciated that the invention is not limited to this particular field of use.

### BACKGROUND

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of the common general knowledge in the field.

Zinc oxide has a number of useful properties, including its UV absorbing, anticorrosion, and antimicrobial properties, that make it suitable for use in a variety of applications. For example, zinc oxide is used in sunscreens, cosmetic formulations, paints, plastics, ceramics, glass, cement, rubber, medicines, foods, and electronic components.

For zinc oxide-based sunscreen formulations, the UV absorbance and associated sun-protection factor (SPF) rating generally increases with increasing concentration of zinc oxide. However, it can be challenging to formulate zinc oxide at high concentrations in stable formulations that retain their transparent properties when applied to the skin. Further, some regulatory bodies limit zinc oxide concentration in topical formulations, e.g. up to 25 wt. %. Accordingly, there is a limit to the amount of zinc oxide that can be incorporated into sunscreen formulations, which prevents higher SPF ratings from being achieved with zinc oxide-based sunscreen.

The SPF rating of a zinc oxide based sunscreen may be increased by adding alternative UV-absorbing ingredients. These are typically organic based UV-absorbers which can themselves cause dermatological issues and other toxicity issues. In contrast inorganic UV absorbers, such as zinc oxide, are classed by the US Food and Drug Administration (FDA) as “GRASE”, or “generally recognized as safe and effective”. There is a need to develop zinc oxide-based materials having improved properties, for example, providing improved UV protection, so that lower concentration zinc oxide-based formulations are able to produce higher SPF rated sunscreen without the need for additional organic based UV-absorbers. There is also a need to develop new zinc oxide-based materials with other advantageous properties that could overcome limitations for other application areas (e.g. improved properties of a sunscreen product, such as improved glow, feel, dryness and antiperspirant properties; or improved composite materials, such as glass composite materials for smart phone device screens having improved crack-resistant properties).

It is an object of the present invention to overcome or ameliorate one or more the disadvantages of the prior art, or at least to provide a useful alternative.

### SUMMARY OF THE INVENTION

The inventor of the present application have surprisingly discovered that by calcination of a mixture of aluminium oxide and zinc carbonate, they are able to produce a calcinated mixture comprising aluminium oxide and zinc oxide having improved UV-absorbing. UV-visible light scattering, and/or UV-visible light reflecting properties over a physical blend of the components.

In a first aspect of the invention there is provided a calcinated mixture comprising aluminium oxide and zinc oxide.

The following options may be used in conjunction with the first aspect, either individually or in any combination.

In one embodiment, the calcinated mixture comprises an aluminium oxide-zinc oxide composite. In certain embodiments, the calcinated mixture comprises an aluminium doped zinc oxide.

The aluminium doped zinc oxide or composite may comprise from about 0.5 at. % to about 10 at. % aluminium, or from about 0.5 at. % to about 5 at. %, about 0.5 at. % to about 2 at. %, about 0.5 at. % to about 1 at. %, about 1 at. % to about 10 at. %, about 1 at. % to about 5 at. %, about 1 at. % to about 2 at. %, or about 0.7 at. % to about 1 at. % aluminium. It may comprise, for example, about 0.5, 1, 1.5, 2, 2.5, 3, 4, 5, 6, 7, 8, 9, or 10 at. % aluminium.

The aluminium doped zinc oxide or composite may be mesoporous. It may have a total mesopore volume of from about 0.2 cm³/g to about 0.9 cm³/g, or from about 0.25 cm³/g to about 0.9 cm³/g, about 0.30 cm³/g to about 0.9 cm³/g, about 0.4 cm³/g to about 0.9 cm³/g, about 0.5 cm³/g to about 0.9 cm³/g, about 0.4 cm³/g to about 0.8 cm³/g, about 0.4 cm³/g to about 0.7 cm³/g, or about 0.4 cm³/g to about 0.6 cm³/g. In certain embodiments, the aluminium doped zinc oxide or composite may have a total mesopore volume of at least about 0.2, 0.25, 0.3, 0.4, 0.45, 0.5, 0.55, 0.6, 0.65, 0.7, 0.75, 0.8, or 0.85 cm³/g. In certain embodiments, the aluminium doped zinc oxide or composite may, for example, have a total mesopore volume of about 0.2, 0.25, 0.3, 0.35, 0.4, 0.45, 0.47, 0.49, 0.5, 0.51, 0.52, 0.55, 0.57, 0.6, 0.65, 0.7, 0.75, 0.8, 0.85, or 0.9 cm³/g. In certain embodiments, the aluminium doped zinc oxide or composite has a total mesopore volume of at least about 0.25 cm³/g. The average pore size in the aluminium doped zinc oxide or composite may be from about 5 nm to about 150 nm, or it may be from about 5 nm to about 100 nm, about 10 nm to about 100 nm, about 20 nm to about 100 nm, about 5 nm to about 80 nm, about 5 nm to about 60 nm, about 20 nm to about 80 nm, about 20 nm to about 60 nm, or about 40 nm to about 60 nm. It may, for example, have an average pore size of about 5, 10, 11, 12, 15, 20, 30, 40, 50, 55, 60, 65, 70, 80, 90, 100, 110, 120, 130, 140, or 150 nm.

In certain embodiments, the aluminium doped zinc oxide or composite may have a specific surface area of from about 10 m²/g to about 100 m²/g, or it may be from about 10 m²/g to about 80 m²/g, about 10 m²/g to about 50 m²/g, about 20 m²/g to about 50 m²/g, about 20 m²/g to about 60 m²/g, about 20 m²/g to about 100 m²/g, about 20 m²/g to about 80 m²/g, about 40 m²/g to about 50 m²/g, about 40 m²/g to about 80 m²/g, about 40 m²/g to about 100 m²/g, or about 40 m²/g to about 60 m²/g. In certain embodiments, the aluminium doped zinc oxide or composite may have a specific surface area of, for example, about 10, 11, 12, 15, 20, 30, 40, 45, 50, 55, 60, 65, 70, 75, 80, 90, or 100 m²/g.

In certain embodiments, the zinc oxide is mesoporous. It may have a total mesopore volume of from about 0.2 cm³/g to about 0.9 cm³/g, or from about 0.25 cm³/g to about 0.9 cm³/g, about 0.30 cm³/g to about 0.9 cm³/g, about 0.4 cm³/g to about 0.9 cm³/g, about 0.5 cm³/g to about 0.9 cm³/g, about 0.4 cm³/g to about 0.8 cm³/g, about 0.4 cm³/g to about 0.7 cm³/g, or about 0.4 cm³/g to about 0.6 cm³/g. In certain embodiments, the zinc oxide may have a total mesopore volume of at least about 0.2, 0.25, 0.3, 0.4, 0.45, 0.5, 0.55, 0.6, 0.65, 0.7, 0.75, 0.8, or 0.85 cm³/g. In certain embodiments, the zinc oxide may, for example, have a total mesopore volume of about 0.2, 0.25, 0.3, 0.35, 0.4, 0.45, 0.47, 0.49, 0.5, 0.51, 0.52, 0.55, 0.57, 0.6, 0.65, 0.7, 0.75, 0.8, 0.85, or 0.9 cm³/g. In certain embodiments, the zinc oxide has a total mesopore volume of at least about 0.25 cm³/g. The average pore size in the zinc oxide may be from about 2 nm to about 100 nm, or it may be from about 5 nm to about 100 nm, about 10 nm to about 100 nm, about 20 nm to about 100 nm, about 2 nm to about 80 nm, about 2 nm to about 60 nm, about 20 nm to about 80 nm, about 20 nm to about 60 nm, or about 40 nm to about 60 nm. It may, for example, have an average pore size of about 2, 5, 10, 11, 12, 15, 20, 30, 40, 50, 55, 60, 65, 70, 80, 90, or 100 nm.

In certain embodiments, the zinc oxide may have a specific surface area of from about 10 m²/g to about 100 m²/g, or it may be from about 20 m²/g to about 100 m²/g, about 20 m²/g to about 80 m²/g, about 40 m²/g to about 100 m²/g, or about 40 m²/g to about 80 m²/g. In certain embodiments, the zinc oxide may have a specific surface area of, for example, about 10, 11, 12, 15, 20, 30, 40, 45, 50, 55, 60, 65, 70, 75, 80, 90, or 100 m²/g.

In certain embodiments, the aluminium oxide may be in the form of particles having a plate-like shape. The aluminium oxide particles may have a mean aspect ratio of at least about 1.5, or at least about 2, 3, 4, 5, 7, 8, or 9. It may be from about 1.5 to about 10, or from about 1.5 to about 8, about 1.5 to about 6, about 1.5 to about 4, about 1.5 to about 2, about 2 to about 5, about 2 to about 4, or about 2 to about 10. It may be about 1.5, 1.6, 1.7, 1.8, 1.9, 1, 1.1, 1.2, 2, 3, 4, 5, 6, 7, 8, 9, or 10. The aspect ratio may be defined as the ratio of the minimum non-thickness dimension to the average thickness. The aluminium oxide particles may be non-uniform in shape, but on average may have non-thickness dimension at least 1.5 times greater than its average thickness. In certain embodiments, the aluminium oxide comprises Alusion™.

The mass ratio of aluminium oxide to zinc oxide in the calcinated mixture may be from about 1:4 to about 1:24, or from about 1:4 to about 1:5, about 1:4 to about 1:10, about 1:4 to about 1:15, about 1:8 to about 1:20, about 1:10 to about 1:20, or about 1:15 to about 1:24. It may be, for example, about 1:4, 1:4.5, 1:5, 1:6; 1:7; 1:8; 1:9; 1:10; 1:12; 1:14; 1:16; 1:18; 1:20; or 1:24. In certain embodiments, the mass ratio of aluminium oxide to zinc oxide in the calcinated mixture is from about 1:4 to about 1:24.

The aluminium oxide may have an average non-thickness dimension of from about 0.1 μm to about 10 μm, or it may be from about 0.1 μm to about 5 μm, about 0.5 μm to about 10 μm, about 0.5 μm to about 10 μm, or about 0.5 μm to about 5 μm. It may be, for example, about 0.1, 0.2, 0.5, 1, 1.1, 1.2, 1.5, 2, 5, or 10 μm. In certain embodiments, the aluminium oxide has an average non-thickness dimension of from about 0.1 to about 50 μm.

The aluminium oxide may have an average thickness of from about 10 nm to about 3000 nm, or it may be from about 10 nm to about 1000 nm, about 10 nm to about 500 nm, about 10 nm to about 250 nm, about 20 nm to about 1000 nm, about 20 nm to about 500 nm, about 20 nm to about 250 nm, about 50 nm to about 1000 nm, about 50 nm to about 500 nm, or about 50 nm to about 250 nm. It may be, for example, about 10, 20, 50, 100, 200, 300, 400, 500, 600, 700, 800, 900, 1000, 2000, or 3000 nm. In certain embodiments, the aluminium oxide has an average thickness of from about 50 to about 250 nm.

In certain embodiments, the calcinated mixture is in the form of a solid powder. The solid powder may have a particle size distribution D₉₀ of from about 1 μm to about 100 μm, or it may be from about 1 μm to about 50 μm, about 1 μm to about 10 μm, about 10 μm to about 50 μm, about 5 μm to about 50 μm, about 10 μm to about 20 μm, about 5 μm to about 10 μm, or about 5 μm to about 25 μm. It may be, for example, about 1, 1.1, 1.2, 1.5, 2, 5, 10, 11, 12, 15, 20, 50, or 100 μm. The solid powder may have a particle size distribution D₅₀ of from about 0.5 μm to about 50 μm, or it may be from about 0.5 μm to about 30 μm, about 0.5 μm to about 10 μm, about 1 μm to about 30 μm, about 5 μm to about 20 μm, about 5 μm to about 10 μm, about 7 μm to about 10.5 μm, or about 5 μm to about 25 μm. It may be, for example, about 0.5, 1, 1.1, 1.2, 1.5, 2, 5, 10, 11, 12, 15, 20, or 50 μm. The solid powder may have a particle size distribution D₁₀ of from about 0.1 μm to about 20 μm, or it may be from about 0.1 μm to about 5 μm, about 0.1 μm to about 10 μm, about 1 μm to about 20 μm, about 1 μm to about 10 μm, about 3 μm to about 6 μm, about 3 μm to about 10 μm, or about 3 μm to about 20 μm. It may be, for example, about 0.1, 0.5, 1, 1.1, 1.2, 1.5, 2, 3, 4, 5, 6, 7, 8, 9, 10, 11, 12, 15, or 20 μm.

In certain embodiments, the calcinated mixture is in the form of a solid powder having the following particle size distribution specifications:
D₁₀: from about 3.0 μm to about 6.0 μm;
D₅₀: from about 7.0 μm to about 10.5 μm; and/or
D₉₀: about 20.0 μm or less.

In a second aspect of the invention there is provided a method to produce a calcinated mixture comprising aluminium oxide and zinc oxide, the method comprising the following steps:
forming a blend of zinc carbonate and aluminium oxide; and
calcinating the blend at a temperature sufficient to convert the zinc carbonate to zinc oxide to thereby form the calcinated mixture.

The following options may be used in conjunction with the second aspect, either individually or in any combination.

In certain embodiments, the aluminium oxide, zinc oxide, and/or calcinated mixture may be as hereinbefore described with respect to the first aspect.

In certain embodiments, the aluminium oxide is in the form of particles having a plate-like shape.

In certain embodiments, the aluminium oxide has an average diameter of from about 0.1 to about 50 μm.

In certain embodiments, the aluminium oxide has an average thickness of from about 50 to about 250 nm.

In certain embodiments, the calcinated mixture comprises an aluminium doped zinc oxide or composite. The aluminium doped zinc oxide or composite may be as hereinbefore described with respect to the first aspect.

In certain embodiments, the zinc carbonate is mesoporous. It may have a total mesopore volume of from about 0.2 cm³/g to about 0.9 cm³/g, or from about 0.25 cm³/g to about 0.9 cm³/g, about 0.30 cm³/g to about 0.9 cm³/g, about 0.4 cm³/g to about 0.9 cm³/g, about 0.5 cm³/g to about 0.9 cm³/g, about 0.4 cm³/g to about 0.8 cm³/g, about 0.4 cm³/g to about 0.7 cm³/g, or about 0.4 cm³/g to about 0.6 cm³/g. In certain embodiments, the zinc carbonate may have a total mesopore volume of at least about 0.2, 0.25, 0.3, 0.4, 0.45, 0.5, 0.55, 0.6, 0.65, 0.7, 0.75, 0.8, or 0.85 cm³/g. In certain embodiments, the zinc carbonate may, for example, have a total mesopore volume of about 0.2, 0.25, 0.3, 0.35, 0.4, 0.45, 0.47, 0.49, 0.5, 0.51, 0.52, 0.55, 0.57, 0.6, 0.65, 0.7, 0.75, 0.8, 0.85, or 0.9 cm³/g. In certain embodiments, the zinc carbonate has a total mesopore volume of at least about 0.25 cm³/g. The average pore size in the zinc carbonate may be from about 2 nm to about 100 nm, or it may be from about 5 nm to about 100 nm, about 10 nm to about 100 nm, about 20 nm to about 100 nm, about 2 nm to about 80 nm, about 2 nm to about 60 nm, about 20 nm to about 80 nm, about 20 nm to about 60 nm, or about 40 nm to about 60 nm. It may, for example, have an average pore size of about 2, 5, 10, 11, 12, 15, 20, 30, 40, 50, 55, 60, 65, 70, 80, 90, or 100 nm.

In certain embodiments, the zinc carbonate may have a specific surface area of from about 10 m²/g to about 100 m²/g, or it may be from about 15 m²/g to about 50 m²/g, about 10 m²/g to about 50 m²/g, about 20 m²/g to about 50 m²/g, about 20 m²/g to about 40 m²/g, or about 20 m²/g to about 30 m²/g. In certain embodiments, the zinc carbonate may have a specific surface area of, for example, about 10, 11, 12, 15, 20, 30, 40, 45, 50, 70, 80, 90, or 100 m²/g.

In certain embodiments, the method may further comprise a step of reacting a zinc halide salt with a carbonate salt to form the zinc carbonate. The zinc halide salt may be zinc chloride. The carbonate salt may be an alkali metal salt, preferably sodium carbonate or potassium carbonate. The step of reacting the zinc halide salt with a carbonate salt may be prior to the forming a blend of zinc carbonate and aluminium oxide step in the method described herein.

In certain embodiments, the method may further comprise a step of reacting a zinc sulfate salt with a carbonate salt to form the zinc carbonate. The carbonate salt may be an alkali metal salt, preferably sodium carbonate or potassium carbonate. The step of reacting the zinc sulfate salt with a carbonate salt may be prior to the forming a blend of zinc carbonate and aluminium oxide step in the method described herein. In certain embodiments, the method further comprises a step of reacting zinc ash with sulfuric acid to form the zinc sulfate salt. The step of reacting the zinc ash with sulfuric acid may be prior to the step of reacting the zinc sulfate salt with the carbonate salt to form the zinc carbonate.

In certain embodiments, the method may further comprise a step of calcinating aluminium hydroxide in the presence of a diluent to form the aluminium oxide. The diluent may preferably be sodium chloride or sodium sulfate. The calcination may be performed at a temperature of about 700° C. or more. The step of calcinating aluminium hydroxide in the presence of a diluent may be prior to the forming a blend of zinc carbonate and aluminium oxide step in the method described herein.

In certain embodiments, the method comprises the following steps:
reacting a zinc halide salt with a carbonate salt to form zinc carbonate;
heating aluminium hydroxide in the presence of a diluent to form aluminium oxide;
forming a blend of the zinc carbonate and aluminium oxide; and
calcinating the blend at a temperature sufficient to convert the zinc carbonate to zinc oxide to thereby form the calcinated mixture.

In certain embodiments, the blend is calcinated at a sufficiently low temperature to retain mesoporosity in the zinc oxide. In certain embodiments, the zinc oxide has a total mesopore volume of at least about 0.25 cm³/g.

The calcinating may be performed in any suitable calcinating apparatus known in the art. For example, the calcinating may be performed using a kiln, such as an electric kiln.

The temperature may be in the range of about 250° C. to about 600° C., about 300° C. to about 600° C., about 350° C. to about 600° C., about 250° C. to about 575° C., about 250° C. to about 550° C., or about 250° C. to about 500° C. It may be, for example, about 250, 300, 350, 400, 450, 500, 550, 575, or 600° C. In certain embodiments, the temperature is in the range of from about 250° C. to about 575° C.

The blend may be calcinated at the temperature for a period of from about 2 hours to about 12 hours, or from about 2.5 hours to about 12 hours, about 2 hours to about 10 hours, about 2 hours to about 8 hours, about 4 hours to about 6 hours; or about 2 hours to about 6 hours. It may be, for example, about 2, 2.5, 3, 3.5, 4, 4.5, 5, 5.5, 6, 7, 8, 9, 10, 11, or 12 hours. In certain embodiments, the blend is calcinated at the temperature for a period of from about 4 hours to about 6 hours. After calcinating at the temperature, the method may further comprise a cooling step, wherein the calcinated mixture is cooled, preferably to room temperature.

The blend may be calcinated to the temperature at a heating rate of from about 5° C./hour to about 500° C./hour, or from about 10° C./hour to about 500° C./hour, about 20° C./hour to about 500° C./hour, about 50° C./hour to about 500° C./hour, about 5° C./hour to about 200° C./hour, about 10° C./hour to about 200° C./hour, about 20° C./hour to about 200° C./hour, about 50° C./hour to about 200° C./hour, or about 50° C./hour to about 150° C./hour. It may be, for example, about 10, 20, 50, 60, 70, 80, 90, 100, 110, 120, 150, 200, 300, 400, or 500° C./hour.

The mass ratio of aluminium oxide to zinc carbonate in the blend may be from about 1:5 to about 1:25, or from about 1:5 to about 1:20, about 1:5 to about 1:10, about 1:5 to about 1:9, about 1:5 to about 1:8, about 1:5 to about 1:7, or about 1:5 to about 1:6. It may be, for example, about 1:5, 1:55, 1:57, 1:6, 1:6.5, 1:7, 1:8, 1:9, 1:10, 1:12, 1:14, 1:16, 1:18, 1:20, 1:22, 1:24, or 1:25. In certain embodiments, the mass ratio of aluminium oxide to zinc carbonate in the blend is from about 1:5 to about 1:25.

In certain embodiments, the method further comprises a communition step. The communition step may comprise grinding or milling the calcinated mixture and/or the blend.

The calcinated mixture according to the first aspect may be produced using the method according to the second aspect. The method of the second aspect may produce the calcinated mixture according to the first aspect.

In a third aspect of the invention there is provided a calcinated mixture comprising aluminium oxide and zinc oxide, which is produced according to the method of the second aspect.

In a fourth aspect of the invention there is provided a composition comprising the calcinated mixture of the first or third aspect.

In certain embodiments, the composition is essentially transparent when applied to a surface, e.g. skin, in the form of a thin film.

In a fifth aspect of the invention there is provided a material comprising the calcinated mixture of the first or third aspect.

In a sixth aspect there is provided a sunscreen formulation comprising the calcinated mixture of the first or third aspect.

The following options may be used in conjunction with the sixth aspect, either individually or in any combination.

In certain embodiments the sunscreen formulation is an organic sunscreen formulation.

In certain embodiments the sunscreen formulation is a vegan sunscreen formulation.

In certain embodiments the sunscreen formulation has a Sun Protection Factor (SPF) rating of about 50 or more.

In a seventh aspect there is provided a method of making a sunscreen formulation, said method comprising mixing the calcinated mixture of the first or third aspect with an oil and/or an aqueous phase.

In certain embodiments the method of the seventh aspect includes the steps of the method of the second aspect.

### BRIEF DESCRIPTION OF THE DRAWINGS

FIG. 1 shows a scanning electron micrograph of aluminium oxide plate-like particles produced in Example 1b.

FIG. 2 shows: (A) reflectance spectra of: (a) calcinated mixture comprising zinc oxide and aluminium oxide; (b) physical blend of zinc oxide and aluminium oxide; (c) zinc oxide; (d) aluminium oxide; and (B) expanded section of FIG. 2(A).

FIG. 3 shows Scanning Electron Microscopy (SEM) & Energy Dispersive Spectroscopy (EDS) of a physical blend of zinc oxide and aluminium oxide:
(A) SEM image of physical blend of zinc oxide and aluminium oxide showing the positions where EDS analyses were performed (Spectra 18-26);
(B) EDS analysis performed at the position indicated as Spectrum 18 in FIG. 3(A);
(C) EDS analysis performed at the position indicated as Spectrum 19 in FIG. 3(A);
(D) EDS analysis performed at the position indicated as Spectrum 20 in FIG. 3(A);
(E) EDS analysis performed at the position indicated as Spectrum 21 in FIG. 3(A);
(F) EDS analysis performed at the position indicated as Spectrum 22 in FIG. 3(A);
(G) EDS analysis performed at the position indicated as Spectrum 23 in FIG. 3(A);
(H) EDS analysis performed at the position indicated as Spectrum 24 in FIG. 3(A);
(I) EDS analysis performed at the position indicated as Spectrum 25 in FIG. 3(A);
(J) EDS analysis performed at the position indicated as Spectrum 26 in FIG. 3(A);
(K) SEM image of physical blend of zinc oxide and aluminium oxide: scale line 100 μm; EHT 20 kV; WD 7.7 mm; Signal A BSD; Aperture Size 60 μm;
(L) SEM image of physical blend of zinc oxide and aluminium oxide: scale line 10 μm; EHT 5 kV; WD 4.8 mm; Signal A InLens; Aperture Size 30 μm;
(M) SEM image of physical blend of zinc oxide and aluminium oxide: scale line 2 μm; EHT 5 kV; WD 4.8 mm; Signal A InLens; Aperture Size 30 μm;
(N) SEM image of physical blend of zinc oxide and aluminium oxide: scale line 1 μm; EHT 5 kV; WD 4.8 mm; Signal A InLens; Aperture Size 30 μm;
(O) SEM image of physical blend of zinc oxide and aluminium oxide: scale line 1 μm; EHT 5 kV; WD 4.8 mm; Signal A SE2; Signal B InLens; Aperture Size 30 μm;
(P) SEM image of physical blend of zinc oxide and aluminium oxide: scale line 200 nm; EHT 5 kV; WD 4.8 mm; Signal A InLens; Aperture Size 30 μm;
(Q) SEM image of physical blend of zinc oxide and aluminium oxide: scale line 200 nm; EHT 5 kV; WD 4.8 mm; Signal A SE2; Signal B InLens; Aperture Size 30 μm;
(R) SEM image of physical blend of zinc oxide and aluminium oxide: scale line 200 nm; EHT 5 kV; WD 4.8 mm; Signal A SE2; Signal B InLens; Aperture Size 30 μm.

FIG. 4 shows Scanning Electron Microscopy (SEM) & Energy Dispersive Spectroscopy (EDS) of a calcinated mixture of zinc oxide and aluminium oxide:
(A) SEM image of calcinated mixture of zinc oxide and aluminium oxide showing the positions where EDS analyses were performed (Spectra 9-17);
(B) EDS analysis performed at the position indicated as Spectrum 9 in FIG. 4(A);
(C) EDS analysis performed at the position indicated as Spectrum 10 in FIG. 4(A);
(D) EDS analysis performed at the position indicated as Spectrum 11 in FIG. 4(A);
(E) EDS analysis performed at the position indicated as Spectrum 12 in FIG. 4(A);
(F) EDS analysis performed at the position indicated as Spectrum 13 in FIG. 4(A);
(G) EDS analysis performed at the position indicated as Spectrum 14 in FIG. 4(A);
(H) EDS analysis performed at the position indicated as Spectrum 15 in FIG. 4(A);
(I) EDS analysis performed at the position indicated as Spectrum 16 in FIG. 4(A);
(J) EDS analysis performed at the position indicated as Spectrum 17 in FIG. 4(A);
(K) SEM image of calcinated mixture of zinc oxide and aluminium oxide: scale line 100 μm; EHT 20 kV; WD 7.6 mm; Signal A BSD; Aperture Size 60 μm;
(L) SEM image of calcinated mixture of zinc oxide and aluminium oxide: scale line 10 μm; EHT 5 kV; WD 4.8 mm; Signal A InLens; Aperture Size 30 μm;
(M) SEM image of calcinated mixture of zinc oxide and aluminium oxide: scale line 2 μm; EHT 5 kV; WD 4.8 mm; Signal A InLens; Aperture Size 30 μm;
(N) SEM image of calcinated mixture of zinc oxide and aluminium oxide: scale line 1 μm; EHT 5 kV; WD 4.8 mm; Signal A InLens; Aperture Size 30 μm;
(O) SEM image of calcinated mixture of zinc oxide and aluminium oxide: scale line 200 nm; EHT 5 kV; WD 4.8 mm; Signal A InLens; Aperture Size 30 μm;
(P) SEM image of calcinated mixture of zinc oxide and aluminium oxide: scale line 1 μm; EHT 5 kV; WD 4.8 mm; Signal A SE2; Signal B InLens; Aperture Size 30 μm;
(Q) SEM image of calcinated mixture of zinc oxide and aluminium oxide: scale line 200 nm; EHT 5 kV; WD 4.8 mm; Signal A SE2; Signal B InLens; Aperture Size 30 μm;
(R) SEM image of calcinated mixture of zinc oxide and aluminium oxide: scale line 200 nm; EHT 5 kV; WD 4.8 mm; Signal A SE2; Signal B InLens; Aperture Size 30 μm.

### DEFINITIONS

In describing and claiming the present invention, the following terminology will be used in accordance with the definitions set out below. It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Unless the context clearly requires otherwise, throughout the description and the claims, the terms “comprise”, “comprising”, and the like are to be construed in an inclusive sense as opposed to an exclusive or exhaustive sense; that is to say, in the sense of “including, but not limited to”. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of.” In other words, with respect to the terms “comprising”, “consisting of”, and “consisting essentially of”, where one of these three terms is used herein, the presently disclosed and claimed subject matter may include the use of either of the other two terms. Thus, in some embodiments not otherwise explicitly recited, any instance of “comprising” may be replaced by “consisting of” or, alternatively, by “consisting essentially of”.

Other than in the operating examples, or where otherwise indicated, all numbers expressing quantities of ingredients or reaction conditions used herein are to be understood as modified in all instances by the term “about”. The examples are not intended to limit the scope of the invention. In what follows, or where otherwise indicated, “%” will mean “weight %”, “ratio” will mean “weight ratio” and “parts” will mean “weight parts”.

The terms “predominantly”, “predominant”, and “substantially” as used herein shall mean comprising more than 50% by weight, unless otherwise indicated.

As used herein, with reference to numbers in a range of numerals, the terms “about,” “approximately” and “substantially” are understood to refer to the range of −10% to +10% of the referenced number, preferably −5% to +5% of the referenced number, more preferably −1% to +1% of the referenced number, most preferably −0.1% to +0.1% of the referenced number. Moreover, with reference to numerical ranges, these terms should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, from 8 to 10, and so forth.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “mesoporous” refers to pores ranging in size from about 2 nm to about 100 nm. The pores are categorized as “open pores” that connect through and open onto a surface of the material or as “closed pores” that are sealed from fluid ingress from the surface of the material. The distribution of pore sizes and total pore volume of the material may be measured using gas adsorption and pycnometry or other techniques which are known to those of skill in the art.

As used herein, the term “calcinated mixture” with respect to the “calcinated mixture comprising zinc oxide and aluminium oxide”, means a mixture comprising zinc oxide and aluminium oxide that has been produced by calcinating a mixture of zinc carbonate and aluminium oxide at a temperature sufficient to convert the zinc carbonate to zinc oxide; that is, the calcinating occurs after the zinc and aluminium complexes are mixed. In certain embodiments, the temperature may be from about 250° C. to about 575° C. In certain embodiments, the “calcinated mixture” may comprise aluminium doped zinc oxide or an aluminium oxide-zinc oxide composite.

As used herein, the term “carbonate” with respect to “zinc carbonate”, includes any carbonate-containing zinc salts. For example, “zinc carbonate” includes ZnCO₃, Zn₅(OH)₆(CO₃)₂, and Zn(HCO₃)₂.

As used herein, the term “at. %” refers to atomic percentage.

Preferred features, embodiments and variations of the invention may be discerned from the following Examples which provides sufficient information for those skilled in the art to perform the invention. The following Examples are not to be regarded as limiting the scope of the preceding Summary of the Invention in any way.

### EXAMPLES

### Example 1: Formation of Aluminium Oxide

The aluminium oxide used herein can be sourced commercially. Alternatively, it may be synthesised. Example procedures for synthesising aluminium oxide are described below.

### Example 1a: Al(OH)₃ in Na₂SO₄ Diluent, Stirred During Heat Treatment

180 g aluminium hydroxide as a precursor compound and 820 g sodium sulphate as a diluent were milled for 1 hour at 400 rpm in a 7 litre attrition mill using 25 kg of 6.35 mm diameter stainless steel balls to form nano-sized particles of aluminium hydroxide. 900 g of the resulting powder was added to a 4 litre alumina crucible containing 2.27 kg of pre-molten sodium sulphate at 1100° C. The mixture was mechanically stirred at 60 rpm by two alumina stirring rods during addition of the milled powder and for a further hour whilst the mixture was held at 1100° C.

X-ray diffraction and electron microscopy studies confirmed that the resulting material consisted of platelets of alpha alumina 0.5-3 microns in diameter with thickness 50-100 nm. The plate-like alumina particles were substantially discrete.

### Example 1b: Al(OH)₃ in NaCl Diluent, with Mineraliser, Solid State Heat Treatment Leading to 0.5-5 Micron Plate-Like Particles

1.04 g aluminium hydroxide as a precursor was milled with 6.88 g NaCl as a diluent and 0.08 g cryolite (Na₃AlF₆) as a mineraliser for 3 hours in a Spex mixer mill using 80 g of 12.7 mm diameter stainless steel balls to form nano-sized particles of aluminium hydroxide. Cryolite is known to be soluble in sodium chloride, forming a eutectic diluent mineraliser system, with eutectic temperature 730° C.

A sample of the resulting powder was heat treated at 500° C. for 30 minutes and washed in deionised water, in order to examine the particle size prior to transformation to alpha phase. X-ray diffraction measurements confirmed that the resulting material was gamma alumina. Electron microscopy studies revealed that the particles were equiaxed nano particles approximately 5 nm in size. The remaining powder was heat treated for 2 hours below the eutectic temperature, at 720° C.

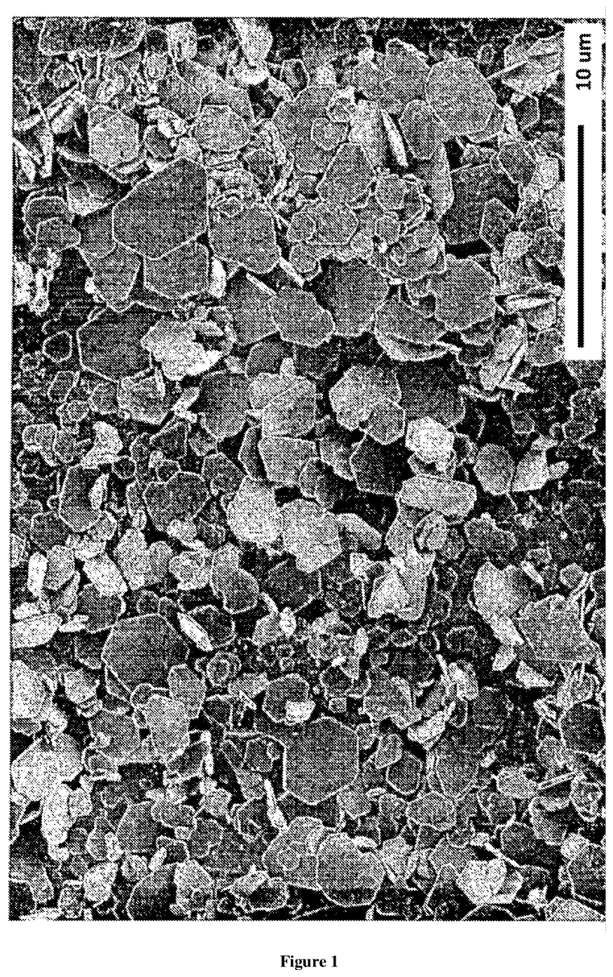

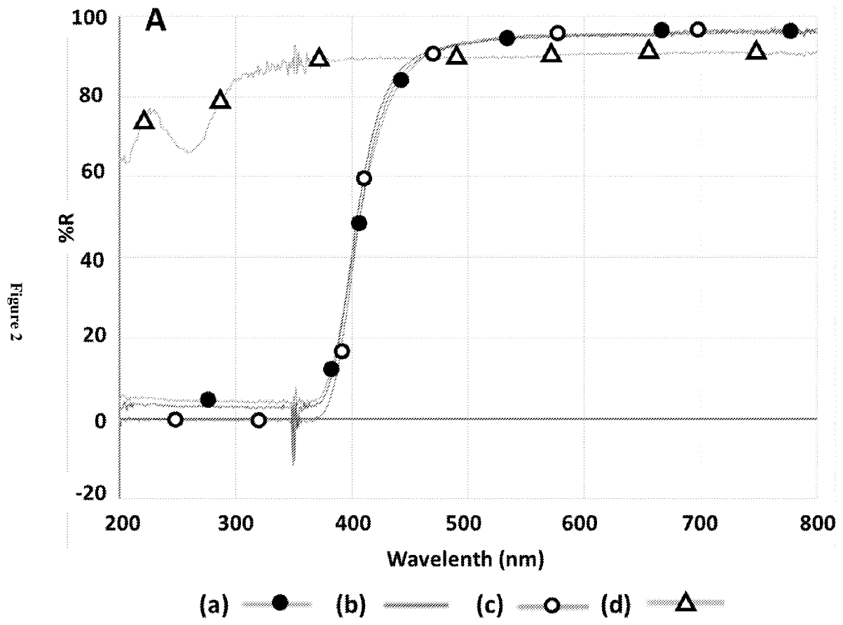

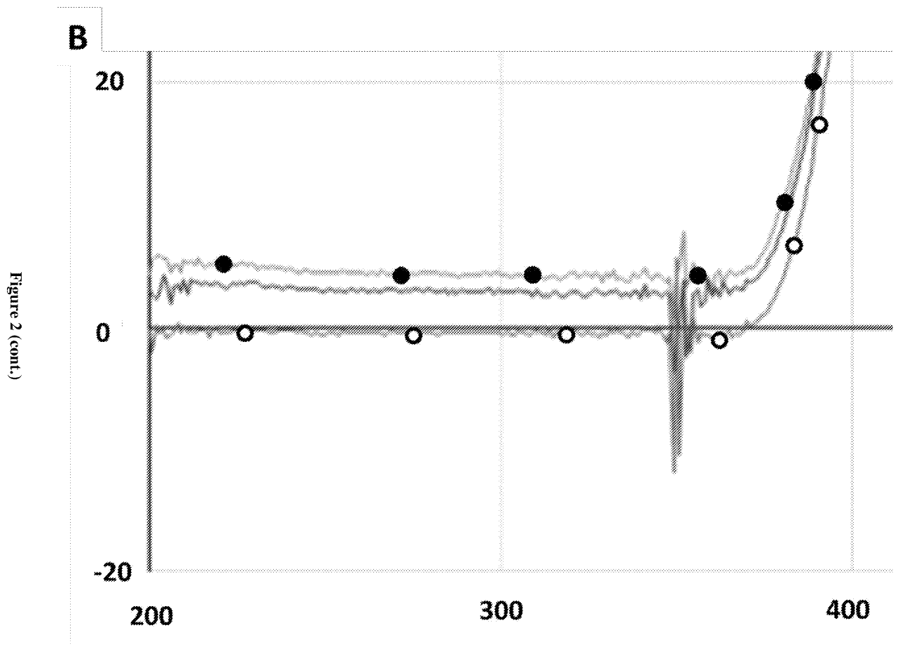

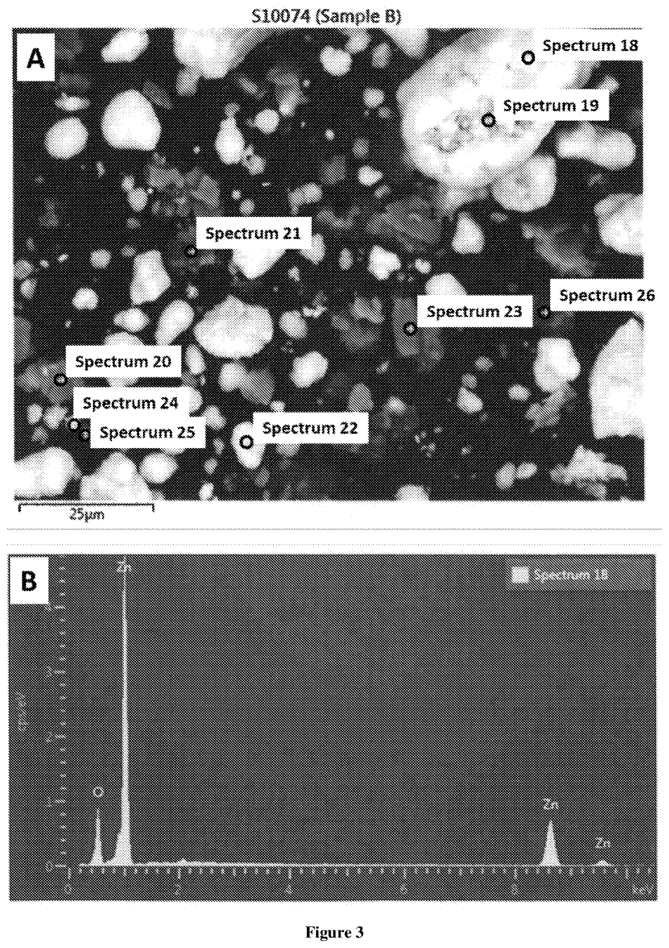

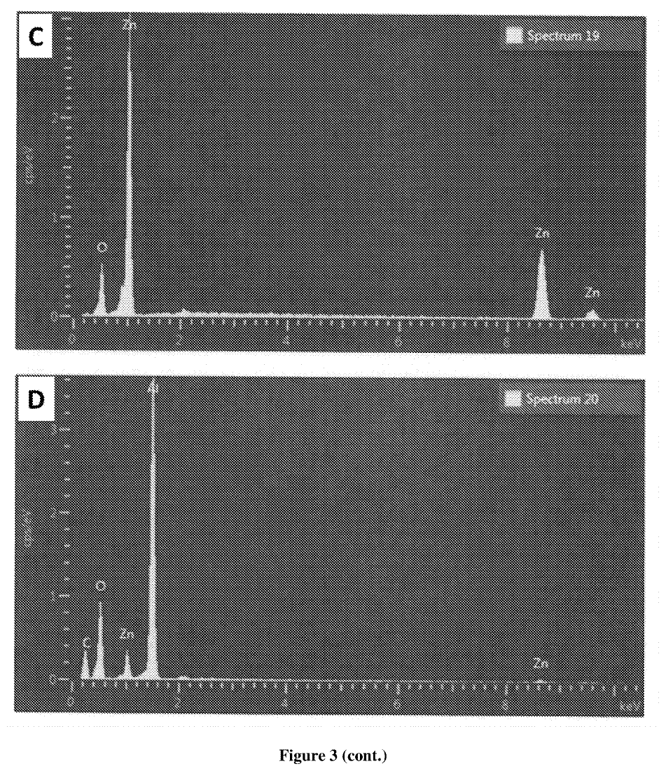

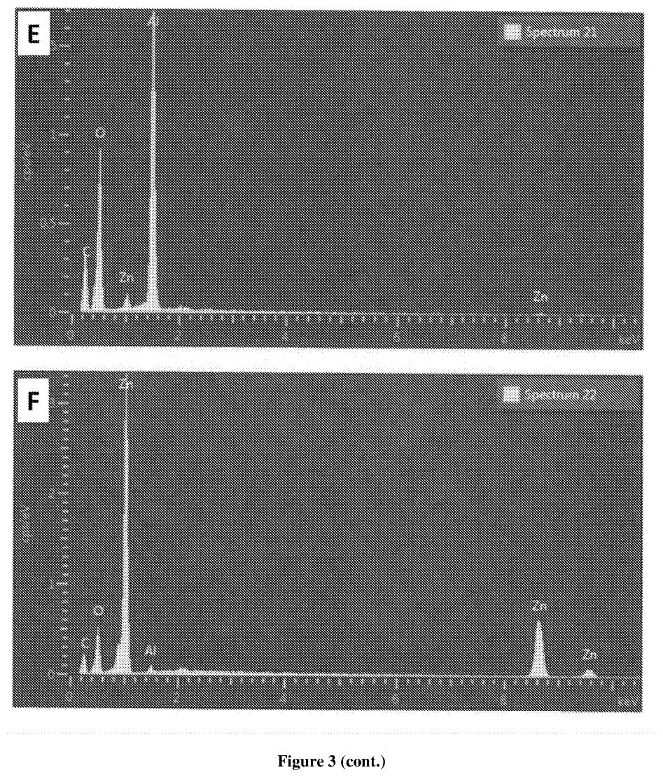

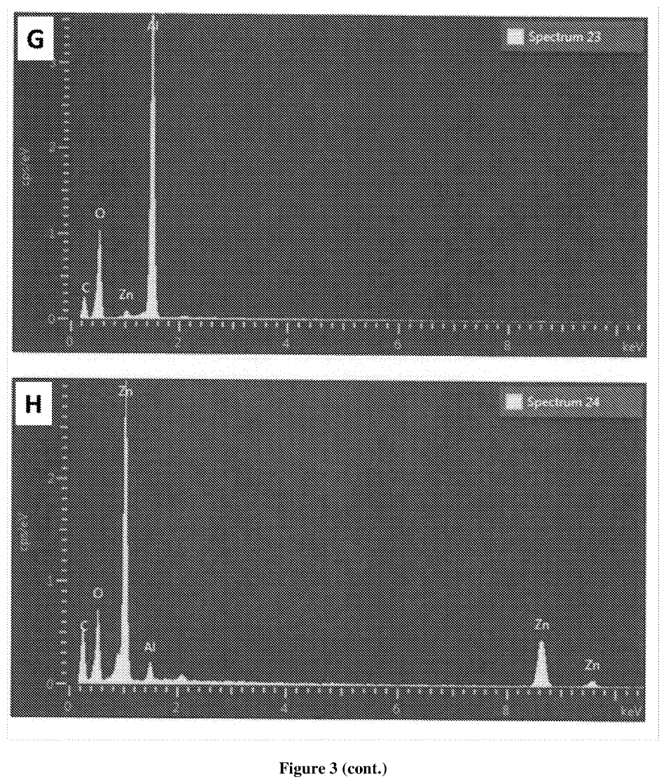

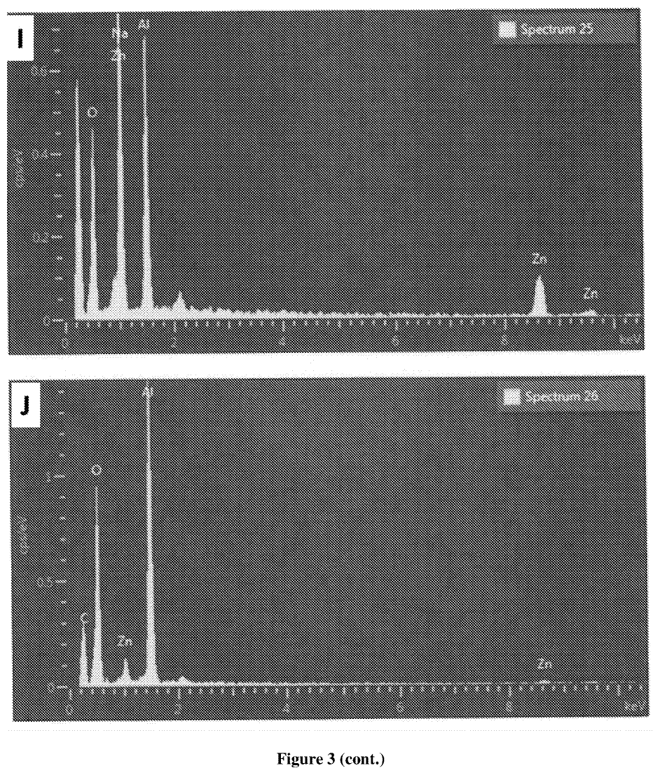

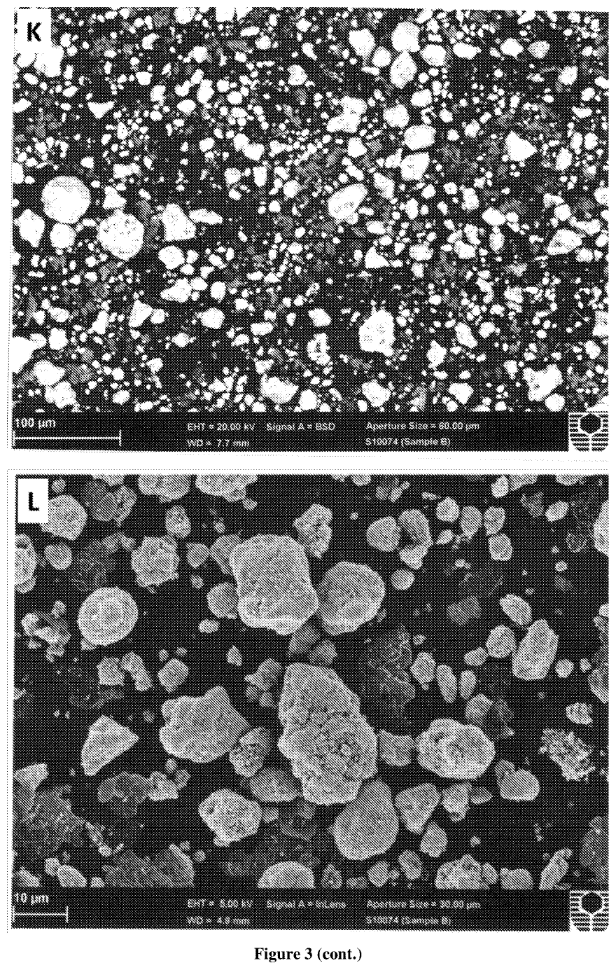

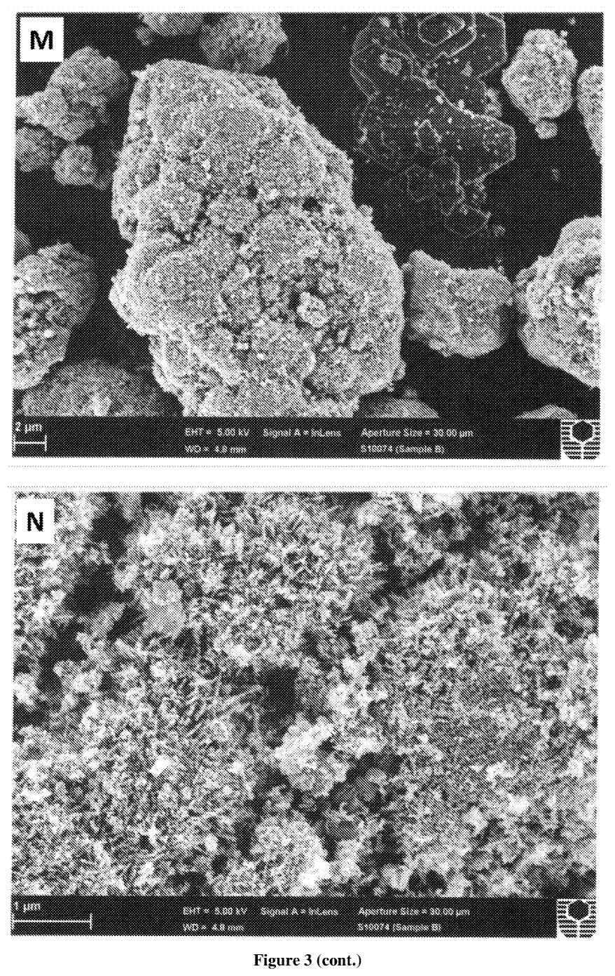

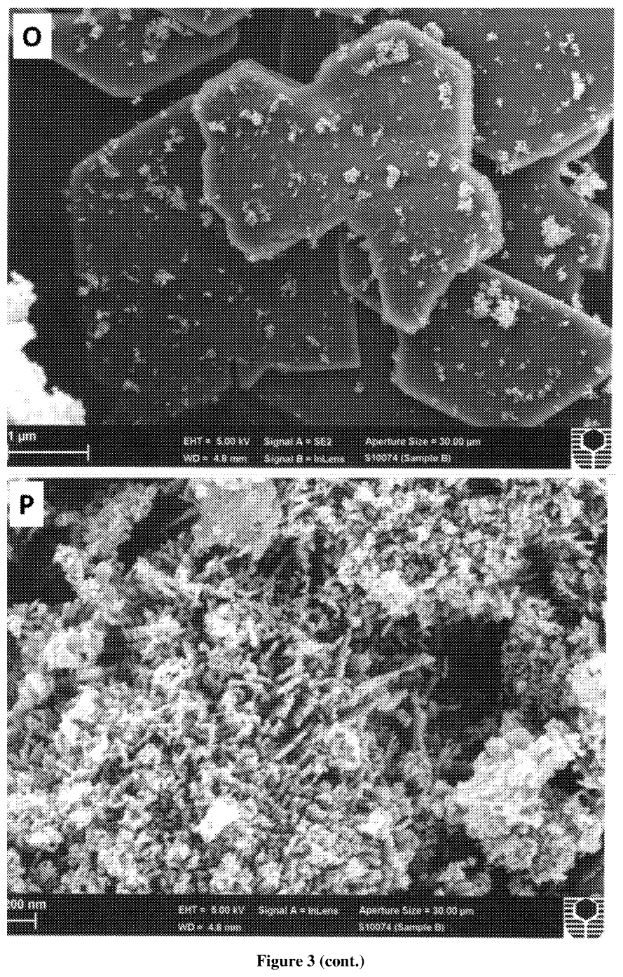

X-ray diffraction and electron microscopy studies confirmed that the resulting material consisted of platelets of alpha alumina 0.5-5 microns in diameter with thickness 50-100 nm. The particles were essentially individual platelets with low levels of agglomeration. FIG. 1 shows a scanning electron micrograph of the material produced using Example 1b.

### Example 1c: Al(OH)₃ in NaCl Diluent, with Mineraliser, Solid State Heat Treatment Below Liquidus Temperature Leading to 0.1-9 Micron Plate-Like Particles

500 g aluminium hydroxide as a precursor compound was milled with 4450 g sodium chloride as a diluent and 50 g cryolite (Na₃AlF₆) as a mineraliser for 90 minutes in a 33 litre attrition mill at 270 rpm, using 100 kg of 6.35 mm diameter stainless steel balls to form nano-sized particles of aluminium hydroxide. The liquidus temperature for diluent-mineraliser system composition is 795-800° C. The resulting powder was heat treated for 2 hours at 780° C.

X-ray diffraction and electron microscopy studies confirmed that the resulting material consisted of plate-like particles of alpha alumina 0.1-9 microns in diameter with thickness 50-150 nm. The substantially discrete particles were essentially individual platelets with low levels of aggregation.

### Example 1d: Partially Dehydrated Al(OH)₃ in NaCl Diluent with Mineraliser, Heat Treatment Below Liquidus Temperature Leading to 1-30 Micron Plate-Like Particles

650 g aluminium hydroxide as a precursor compound which had been dried to 23% mass loss at 230° C. was milled with 4300 g sodium chloride as a diluent and 50 g cryolite as a mineraliser for 90 minutes at 270 rpm in a 33 litre attrition mill using 100 kg of 6.35 mm diameter stainless steel balls to form an intermediate compound comprising nano-sized particles of aluminium hydroxide.

X-ray diffraction measurements showed that the starting hydroxide material was predominantly boehmite (AlOOH), with a small fraction of gibbsite (Al(OH)₃) remaining. The resulting powder was heat treated for 2 hours at 780° C.

X-ray diffraction and electron microscopy studies confirmed that the resulting material consisted of platelets of alpha alumina 1-30 microns in diameter with thickness 50-200 nm. The particles were essentially individual platelets with low levels of aggregation.

### Example 2: Preparation of Mesoporous Zinc Carbonate Precursor

Zinc carbonate precursor powder was synthesized by reacting aqueous solutions of zinc chloride and sodium carbonate in the molar ratio of 1ZnCl₂:3Na₂CO₃ at room temperature. The individual solutions consisted of 1230 g of zinc chloride dissolved in 4 L of deionized (DI) water and 960 g of sodium carbonate dissolved in 10 L of DI water. The zinc chloride solution was added under vigorous stirring to the carbonate solution resulting in a white precipitate. The precipitate was washed using deionized water to less than 100 ppm and dried at 120° C.

The crystal structure of the resulting powder was characterized by x-ray diffraction which showed the hydrozincite phase as the only phase present. Scanning electron microscope (SEM) examination of the powder showed that it consisted of mesoporous aggregates of primary crystallites. The specific surface area of the powder measured using gas adsorption (BET method, Micromeritics Tristar) was 62.4 m²/g.

The distribution of open pores was measured using gas adsorption techniques (Micromeritics Tristar) according to the Barrett-Joyner-Helenda method (described in Techniques de l'Ingenieur [Techniques of the Engineer] and entitled “Texture des solides poreux ou divises” [Texture of porous or divided solids], p. 3645-1 to 3645-13). The pore size measurements showed a distribution of pore sizes between 2 nm and 100 nm (mesopores) with the average pore size equal to 27.3 nm. The total open mesopore volume was 0.476 cm³/g.

### Example 3: Preparation of Calcinated Mixture of Zinc Oxide and Aluminium Oxide

Aluminium oxide (Alusion™) and the mesoporous zinc carbonate precursor were mixed in a mass ratio of 0.04-0.17 aluminium oxide:0.83-0.96 zinc carbonate. The mixture was heat treated at a temperature of from about 385° C. to about 500° C. in an electric kiln. The samples were subject to slow heating with a furnace ramp rate of form about 50° C./hr to about 150° C./hr and held for from about 3 hours to about 8 hours at the set temperature to convert the zinc carbonate to zinc oxide, followed by cooling to room temperature.

### Example 4: Comparative Preparation of Calcinated Zinc Oxide without Aluminium Oxide

Zinc oxide powder was prepared from the hydrozincite powder of Example 2 by heat treating at a temperature of from about 385° C. to about 500° C. in an electric kiln. The samples were subject to slow heating with a furnace ramp rate of from about 50° C./hr to about 150° C./hr and held for from about 3 hours to about 8 hours at the set temperature, followed by cooling to room temperature. The resulting powder had an off-white colour. X-ray diffraction showed that ZnO (wurtzite phase) was the only crystalline phase present after calcining.

### Example 5: Comparison of Physical Blend of Zinc Oxide and Aluminium Oxide with Calcinated Mixture of Zinc Oxide and Aluminium Oxide

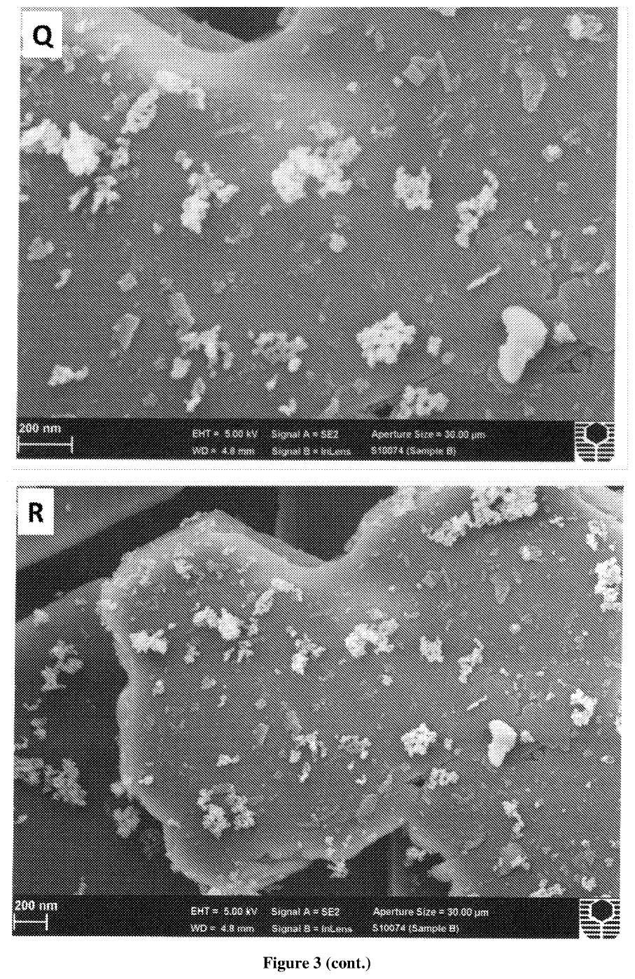

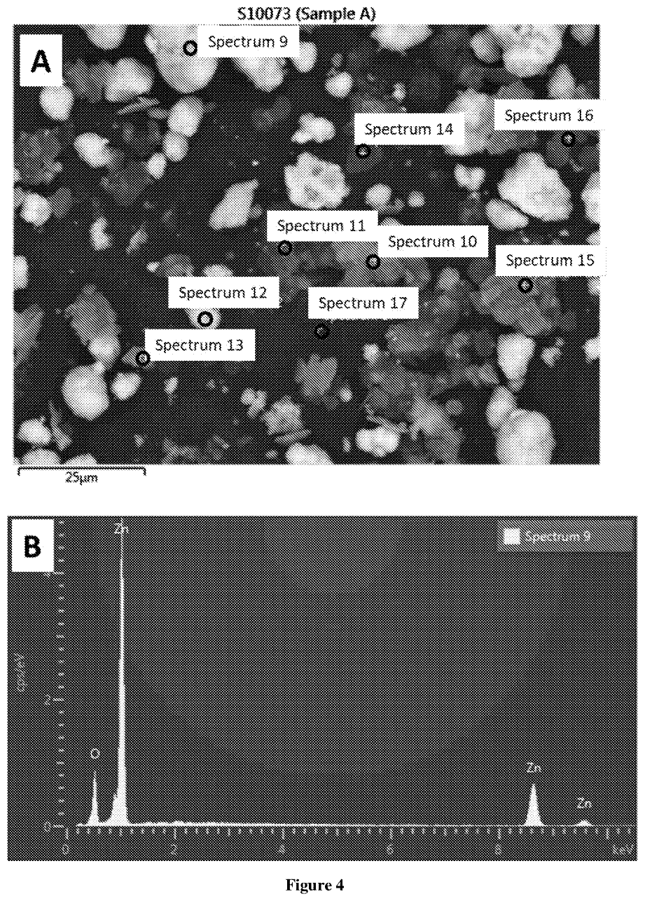

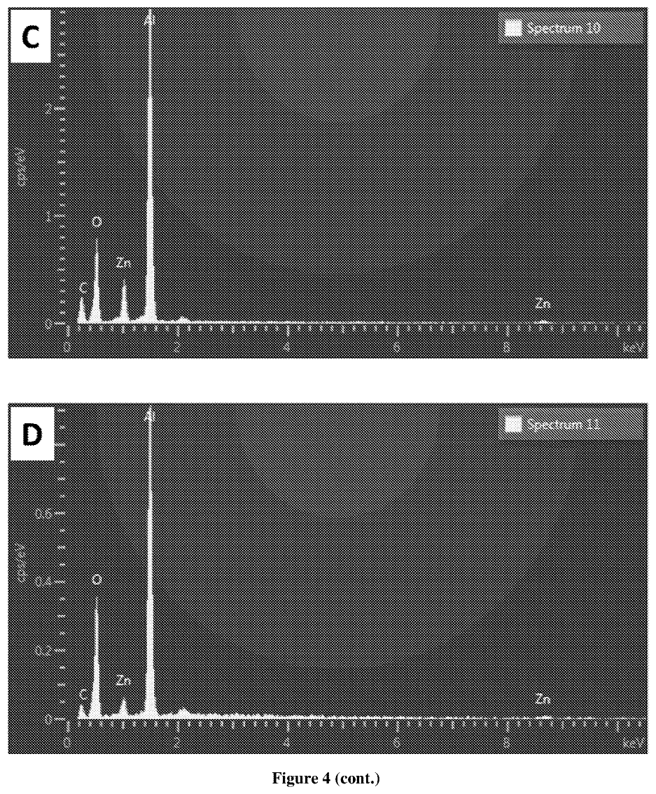

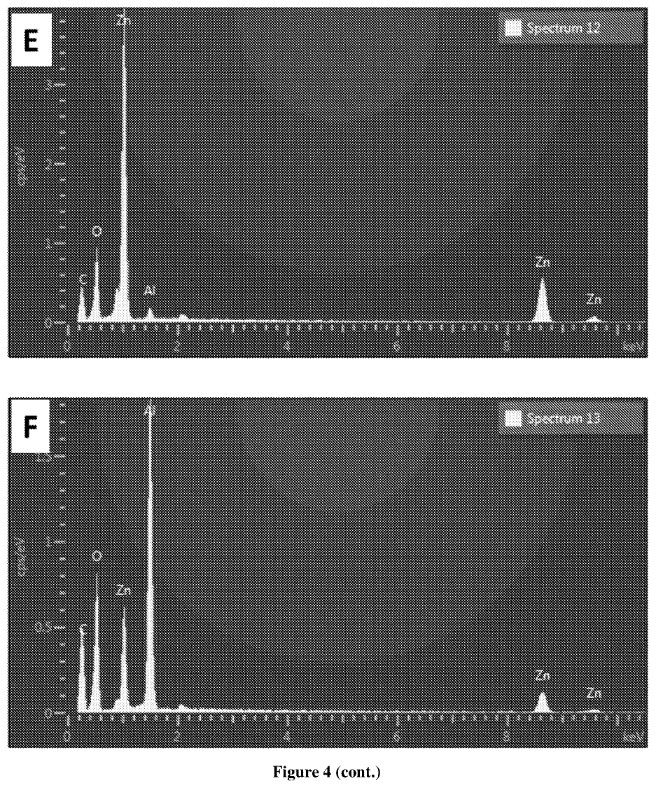

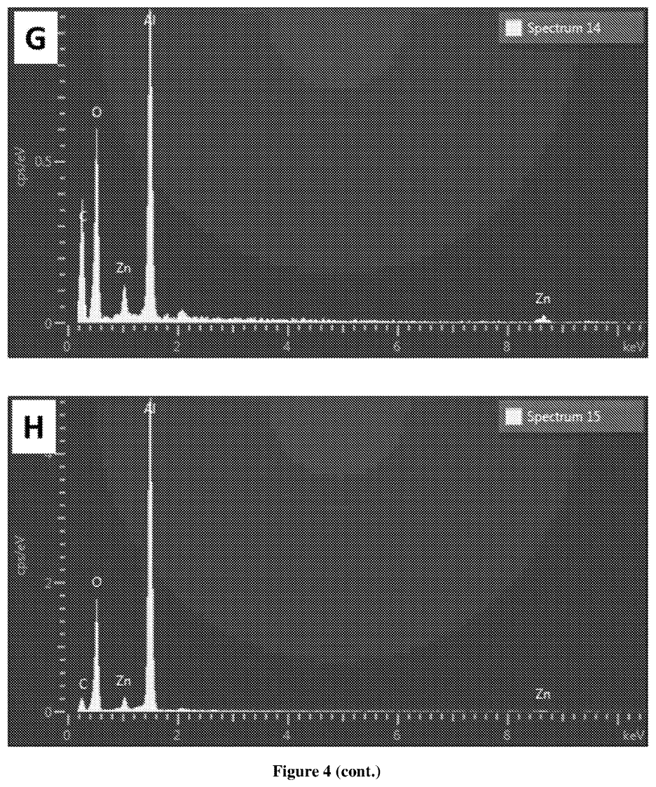

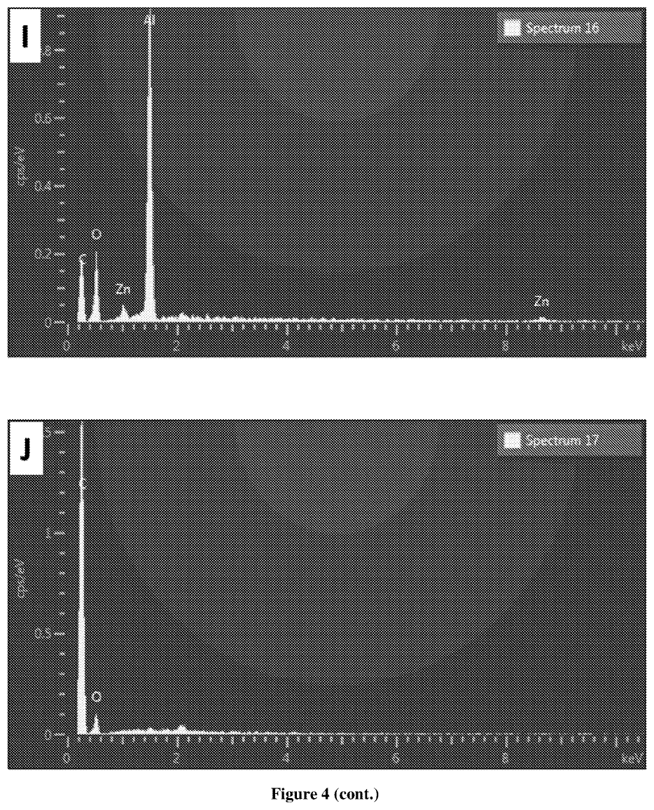

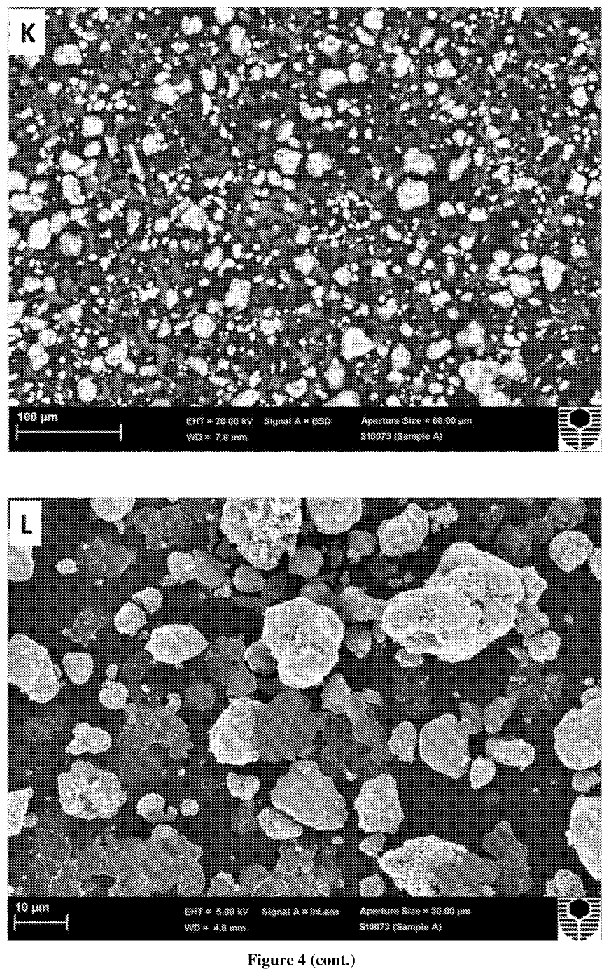

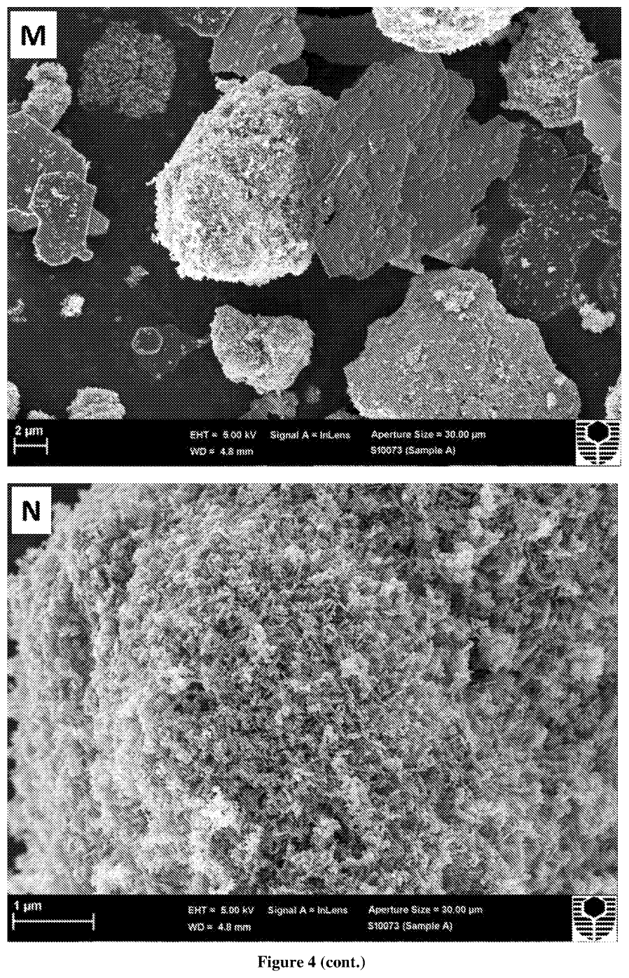

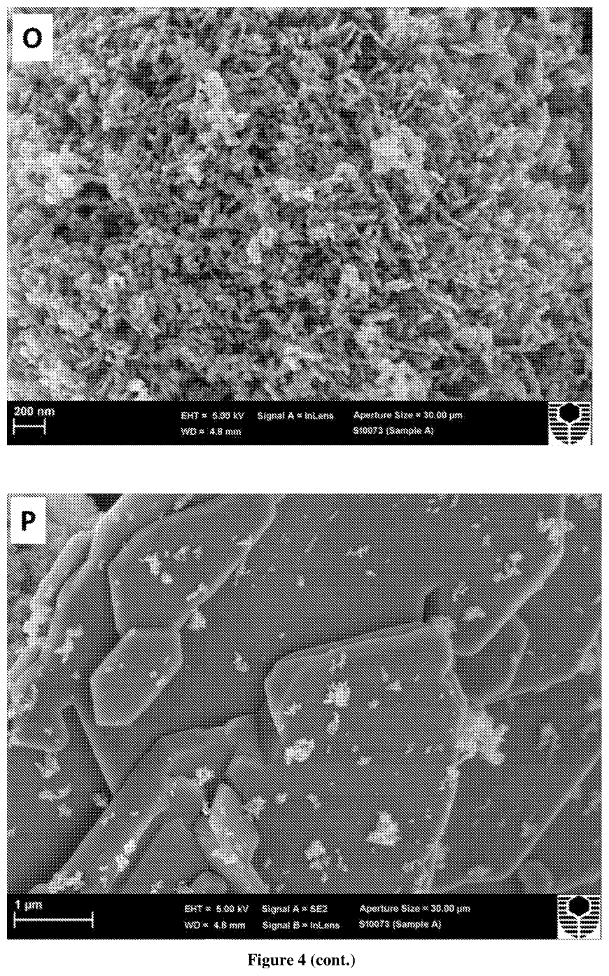

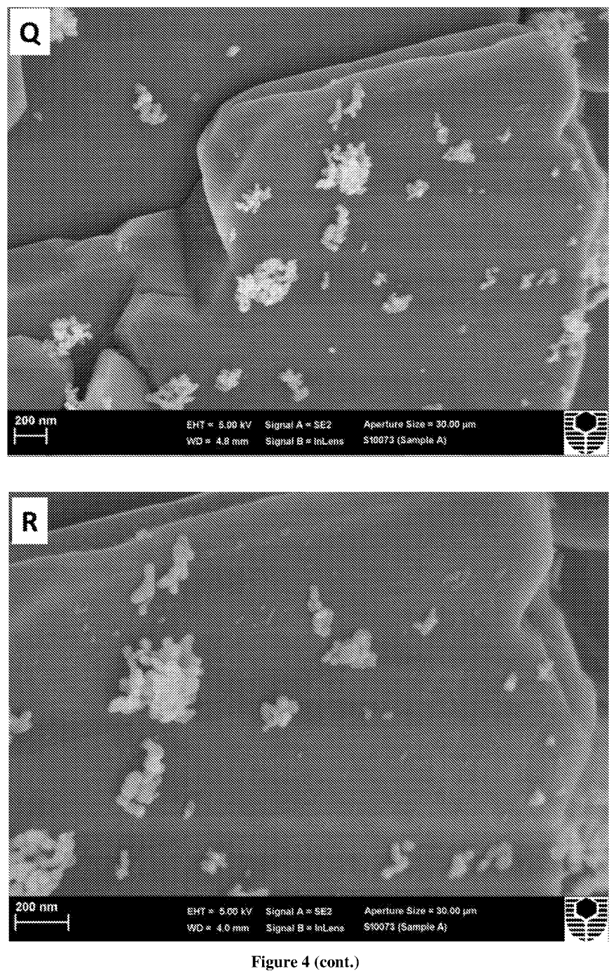

UV-visible reflectance spectroscopy shows that a calcinated mixture of zinc oxide and aluminium oxide has a higher reflectance in the UV region as compared with a physical blend of zinc oxide and aluminum oxide having the same stochiometric ratio of zinc oxide to aluminium oxide (FIG. 2).

SEM and EDS analyses of a physical blend of zinc oxide and aluminium oxide are shown in FIG. 3. FIG. 4 shows SEM and EDS analyses of a calcinated mixture of zinc oxide and aluminium oxide.

### Example 6: Preparation of Formulations Containing Calcinated Mixture of Zinc Oxide and Aluminium Oxide

The calcinated mixture of zinc oxide and aluminium oxide was incorporated in a variety of water-in-oil emulsion-based sunscreen formulations, by mixing the calcinated mixture with the other formulation components in a heated mixture, typically at around 40-80° C.

A variety of different sunscreen formulations were prepared, including formulations with equivalent amounts (by mass) of:
(1) zinc oxide only (made according to the method outlined above at Example 4);
(2) a physical blend of zinc oxide (made according to the method outlined above at Example 4) and aluminium oxide (made according to the method outlined above at Example 1); or
(3) the inventive calcinated mixture of zinc oxide and aluminium oxide (made according to the method outlined above at Example 3).

The components and amounts for the formulations comprising zinc oxide and aluminium oxide (i.e. (2) and (3)) are set out in the table below:

| Ingredient Name | wt. % Range |
|---|---|
| Zinc Oxide | 20.0-25.0 |
| Aluminium Oxide | 3.0-10.0 |
| Helianthus Annuus Seed Oil | 15.0-25.0 |
| Simmondsia Chinensis (Jojoba) Seed oil | 2.0-6.0 |
| Polyglyceryl-3 Polyricinoleate | 1.0-5.0 |
| Polyhydroxy Stearic Acid | 0.5-1.0 |
| Euphorbia Cerifera (Candelilla) Wax | 1.0-5.0 |
| Isostearic Acid | 0.5-2.0 |
| Xanthan Gum | 0.5-2.0 |
| Sodium Chloride | 0.5-2.0 |
| Glycerin | 10.0-15.0 |
| Maltodextrin | 0.5-2.0 |
| Potassium Cetyl Phosphate | 0.5-2 |
| Water | Up to 100 |

These formulations were tested for their SPF (sun proof factor) rating to determine the effect of the calcinating process on the SPF rating of the resultant formulation. SPF ratings for the formulations are shown in table 1 below.

TABLE 1
SPF results for sunscreen formulations containing: (1) zinc
oxide only; (2) a physical blend of zinc oxide and aluminium
oxide; or (3) the inventive calcinated mixture of zinc oxide
and aluminium oxide. Formulations grouped in the same cell
have equivalent amounts (by mass) of zinc oxide (in the case
of (1)), or total zinc oxide and aluminium oxide (in the
case of (2) or (3)), but are otherwise identical.

| | SPF rating | | |
|---|---|---|---|
| | | | (3) |
| | | (2) | Calcinated |
| Formulation | (1) | Physical blend | mixture |
| No. | Zinc oxide only | ZnO + Al₂O₃ | ZnO + Al₂O₃ |
| 1 | Not determined | 16 | 30 |
| 2 | 15.3 | 27 | 35 |
| 3 | 26.6 | 31 | 54 |

The SPF results for the formulations tested indicate a significant increase in SPF rating for sunscreen formulations containing the calcinated mixture of zinc oxide and aluminium oxide as compared with the physical blend of zinc oxide and aluminium oxide, and zinc oxide only formulations.

Without being bound by theory, the inventor of the present application postulate that by calcinating (heating) the zinc carbonate (to form zinc oxide) in the presence of aluminium oxide, that some of the aluminium atoms may be incorporated into, or dope the zinc oxide, resulting in a new material with improved UV absorption, UV-visible light scattering, and/or UV-visible light reflecting properties.

Although the invention has been described with reference to specific examples, it will be appreciated by those skilled in the art that the invention may be embodied in many other forms. In particular, features of any one of the various described examples may be provided in any combination in any of the other described examples. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A calcinated mixture comprising aluminium oxide and zinc oxide.

2. The calcinated mixture of claim 1, comprising an aluminium doped zinc oxide or an aluminium oxide-zinc oxide composite.

3. The calcinated mixture of claim 1, wherein the zinc oxide is mesoporous optionally wherein the zinc oxide has a total mesopore volume of at least about 0.25 cm³/g.

4. (canceled)

5. The calcinated mixture of claim 1, wherein the aluminium oxide has a plate-like shape.

6. The calcinated mixture of claim 1, wherein the mass ratio of aluminium oxide to zinc oxide in the calcinated mixture is from about 1:4 to about 1:24.

7. The calcinated mixture of claim 1, wherein the aluminium oxide has an average non-thickness dimension of from about 0.1 to about 10 μm.

8. The calcinated mixture of claim 1, which is in the form of a solid powder.

9. A method to produce a calcinated mixture comprising aluminium oxide and zinc oxide, the method comprising the following steps:
forming a blend of zinc carbonate and aluminium oxide; and
calcinating the blend at a temperature sufficient to convert the zinc carbonate to zinc oxide to thereby form the calcinated mixture.

10. The method of claim 9, wherein the aluminium oxide has a plate-like shape.

11. The method of claim 9, wherein the aluminium oxide has an average non-thickness dimension of from about 0.1 to about 10 μm.

12. The method of claim 9, wherein the calcinated mixture comprises an aluminium doped zinc oxide or an aluminium oxide-zinc oxide composite.

13. The method of claim 9, wherein the zinc carbonate is mesoporous.

14. The method of claim 13, wherein the zinc carbonate has a total mesopore volume of at least about 0.25 cm³/g.

15. The method of claim 13, wherein the blend is calcinated at a sufficiently low temperature to retain mesoporosity in the zinc oxide.

16. The method of claim 15, wherein the zinc oxide has a total mesopore volume of at least about 0.25 cm³/g.

17. The method of claim 9, wherein the temperature is in the range of from about 250° C. to about 575° C.

18. The method of claim 9, wherein the blend is calcinated at the temperature for a period of from about 4 hours to about 6 hours.

19. The method of claim 9, wherein the mass ratio of aluminium oxide to zinc carbonate in the blend is from about 1:5 to about 1:25.

20. The method of claim 9, further comprising a communition step.

21. (canceled)

22. A composition comprising the calcinated mixture of claim 1.

23. (canceled)
